(19) United States
(12) Patent Application Publication
(10) Pub. No.: US 2024/0170117 A1
(43) Pub. Date: May 23, 2024

(54) Homeowner Health Alerts and Mitigation Based on Home Sensor Data

(71) Applicant: STATE FARM MUTUAL AUTOMOBILE INSURANCE COMPANY, Bloomington, IL (US)

(72) Inventors: Eric Allyn Finley, Bloomington, IL (US); Jennifer L. Crawford, Normal, IL (US); Corin Rebekah Chapman, Bloomington, IL (US); Edward W. Breitweiser, Bloomington, IL (US); Gregory Wong, New Albany, OH (US)

(21) Appl. No.: 18/099,570

(22) Filed: Jan. 20, 2023

Related U.S. Application Data
(60) Provisional application No. 63/428,723, filed on Nov. 29, 2022.
(60) Provisional application No. 63/427,596, filed on Nov. 23, 2022.
(60) Provisional application No. 63/427,495, filed on Nov. 23, 2022.
(60) Provisional application No. 63/427,680, filed on Nov. 23, 2022.

(51) Int. Cl. G16H 20/00; G08B 21/04; G16H 40/67; G16H 50/20

(52) U.S. Cl. CPC G16H 20/00; G08B 21/0484; G16H 40/67; G16H 50/20

(57) ABSTRACT

Techniques for detecting health conditions associated with residents of a home environment based upon data captured by in-home sensors may include monitoring sensor data associated with a home environment; analyzing the sensor data over a first period of time in order to identify a health condition associated with a resident of the home environment; identifying one or more mitigation techniques for the health condition; providing an indication of the one or more mitigation techniques for the health condition, via a user interface; and analyzing the sensor data associated with the home environment over a second period of time, subsequent to providing the indication of the one or more mitigation techniques for the health condition via the user interface, in order to determine whether any of the one or more mitigation techniques were performed.

### CROSS-REFERENCE TO RELATED APPLICATIONS

The present application claims priority to U.S. Provisional Patent Application No. 63/427,596, entitled “Homeowner Health Alerts and Mitigation based on Home Sensor Data,” and filed Nov. 23, 2022; U.S. Provisional Patent Application No. 63/428,723, entitled “Homeowner Health Alerts and Mitigation based on Home Sensor Data,” and filed Nov. 29, 2022;” U.S. Provisional Patent Application No. 63/427,495, entitled “Home Condition Alerts based on Home Sensor Data,” and filed Nov. 23, 2022; and U.S. Provisional Patent Application No. 63/427,680, entitled “Home and Vehicle Repair Diagnostics,” and filed Nov. 23, 2022; the disclosures of each of which are incorporated by reference herein.

### FIELD OF THE INVENTION

The present disclosure generally relates to technologies associated with detecting health conditions, more particularly, to technologies for detecting health conditions associated with residents of a home environment based upon data captured by in-home sensors.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

It may sometimes be difficult for individuals to identify health conditions. Some individuals may not realize that certain conditions may be health concerns, or may be aware that certain conditions may be health concerns but may not realize that such conditions are developing. Moreover, even when individuals can identify health concerns, they may not be aware of mitigating techniques that may be used to alleviate such health concerns. Conventional techniques may include additional inefficiencies, encumbrances, ineffectiveness, and/or other drawbacks.

### SUMMARY

The present embodiments may relate to, inter alia, technologies associated with detecting health conditions, as well as technologies for detecting health conditions associated with residents of a home based upon data captured by in-home sensors.

In one aspect, a computer-implemented method for detecting health conditions associated with residents of a home environment based upon data captured by in-home sensors may be provided. The method may be implemented via one or more local or remote processors, transceivers, sensors, servers, memory units, mobile devices, wearables, smart watches, smart contact lenses, virtual headsets (e.g., virtual reality (VR) headsets, smart glasses, augmented reality (AR) glasses, mixed or extended glasses or headsets, etc.), and/or other electronic or electric components. In one instance, the method may include (1) monitoring, by one or more processors, sensor data associated with a home environment; (2) analyzing, by one or more processors, the sensor data associated with the home environment over a first period of time in order to identify a health condition associated with a resident of the home environment; (3) identifying, by the one or more processors, one or more mitigation techniques for the health condition associated with the resident of the home environment; (4) providing, by the one or more processors, an indication of the one or more mitigation techniques for the health condition associated with the resident of the home environment, via a user interface; and/or (5) analyzing, by one or more processors, the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time. The method may include additional, less, or alternate actions, including those discussed elsewhere herein.

In another aspect, a computer system for detecting health conditions associated with residents of a home environment based upon data captured by in-home sensors may be provided. The computer system may include one or more local or remote processors, transceivers, sensors, servers, memory units, mobile devices, wearables, smart watches, smart contacts, virtual headsets (e.g., virtual reality (VR) headsets, smart glasses, augmented reality (AR) glasses, mixed or extended reality headsets or glasses, etc.), and/or other electronic or electric components. In one instance, the computer system may include one or more processors and a memory storing computer-executable instructions that, when executed by the one or more processors, cause the one or more processors to: (1) monitor sensor data associated with a home environment; (2) analyze the sensor data associated with the home environment over a first period of time in order to identify a health condition associated with a resident of the home environment; (3) identify one or more mitigation techniques for the health condition associated with the resident of the home environment; (4) provide an indication of the one or more mitigation techniques for the health condition associated with the resident of the home environment, via a user interface; and/or (5) analyze the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time. The system may include additional, less, or alternate functionality, including that discussed elsewhere herein.

In still another aspect, a non-transitory computer-readable storage medium storing computer-readable instructions for detecting health conditions associated with residents of a home environment based upon data captured by in-home sensors may be provided. The computer-readable instructions, when executed by one or more processors, cause the one or more processors to: (1) monitor sensor data associated with a home environment; (2) analyze the sensor data associated with the home environment over a first period of time in order to identify a health condition associated with a resident of the home environment; (3) identify one or more mitigation techniques for the health condition associated with the resident of the home environment; (4) provide an indication of the one or more mitigation techniques for the health condition associated with the resident of the home environment, via a user interface; and/or (5) analyze the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time. The instructions may direct additional, less, or alternative functionality, including that discussed elsewhere herein.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 depicts an exemplary computer system for detecting health conditions associated with residents of a home environment based upon data captured by in-home sensors, according to one embodiment;

FIG. 2 depicts a flow diagram of an exemplary computer-implemented for detecting health conditions associated with residents of a home environment based upon data captured by in-home sensors, according to one embodiment;

FIG. 3 depicts an exemplary computing system in which the techniques described herein may be implemented, according to one embodiment.

While the systems and methods disclosed herein is susceptible of being embodied in many different forms, it is shown in the drawings and will be described herein in detail specific exemplary embodiments thereof, with the understanding that the present disclosure is to be considered as an exemplification of the principles of the systems and methods disclosed herein and is not intended to limit the systems and methods disclosed herein to the specific embodiments illustrated. In this respect, before explaining at least one embodiment consistent with the present systems and methods disclosed herein in detail, it is to be understood that the systems and methods disclosed herein is not limited in its application to the details of construction and to the arrangements of components set forth above and below, illustrated in the drawings, or as described in the examples.

Methods and apparatuses consistent with the systems and methods disclosed herein are capable of other embodiments and of being practiced and carried out in various ways. Also, it is to be understood that the phraseology and terminology employed herein, as well as the abstract included below, are for the purposes of description and should not be regarded as limiting.

### DETAILED DESCRIPTION

Using the techniques provided herein, data captured by sensors that may already be implemented by home security systems, home monitoring systems, or other smart home systems may be analyzed in order to, inter alia, identify health conditions associated with residents of the home. Alerts may be generated to indicate possible health conditions that are detected. In some cases, the alert may be provided to an emergency contact designated by the resident of the home. Moreover, for very severe/urgent alerts, emergency services may be contacted. Additionally, the alerts may indicate mitigation steps that users can take based upon possible health conditions detected. The sensor data may be analyzed further in order to identify whether any identified mitigation steps have been taken by the resident of the home environment, and/or whether any previously-identified health conditions have changed. In some cases, insurance premiums may be reduced based upon the resident of the home taking the identified mitigation steps.

Exemplary System for Detecting Health Conditions Associated with Residents of a Home Environment Based Upon Data Captured by In-Home Sensors

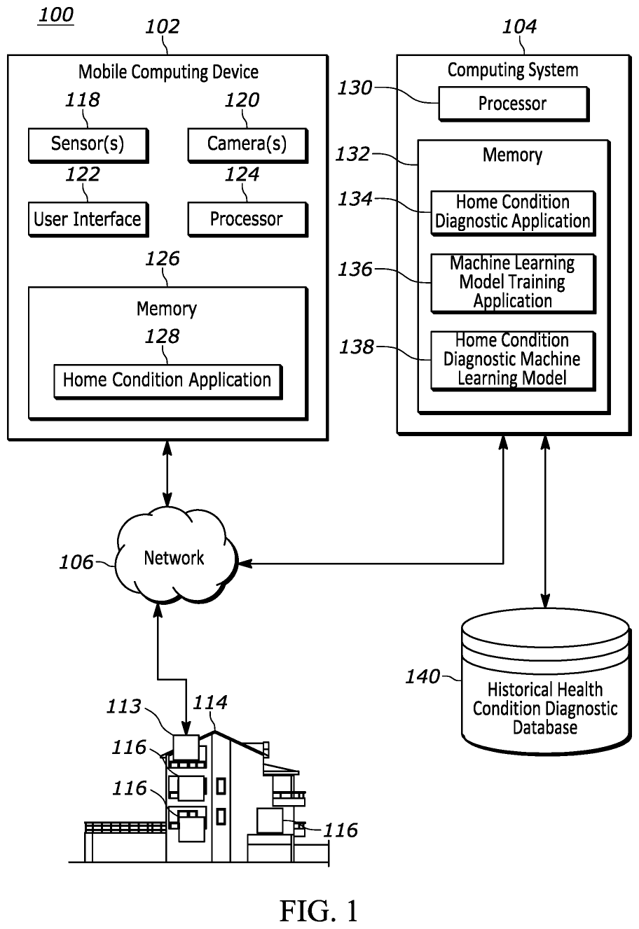

Referring now to the drawings, FIG. 1 depicts an exemplary computer system 100 for detecting health conditions associated with residents of a home based upon data captured by in-home sensors, according to one embodiment. The high-level architecture illustrated in FIG. 1 may include both hardware and software applications, as well as various data communications channels for communicating data between the various hardware and software components, as is described below.

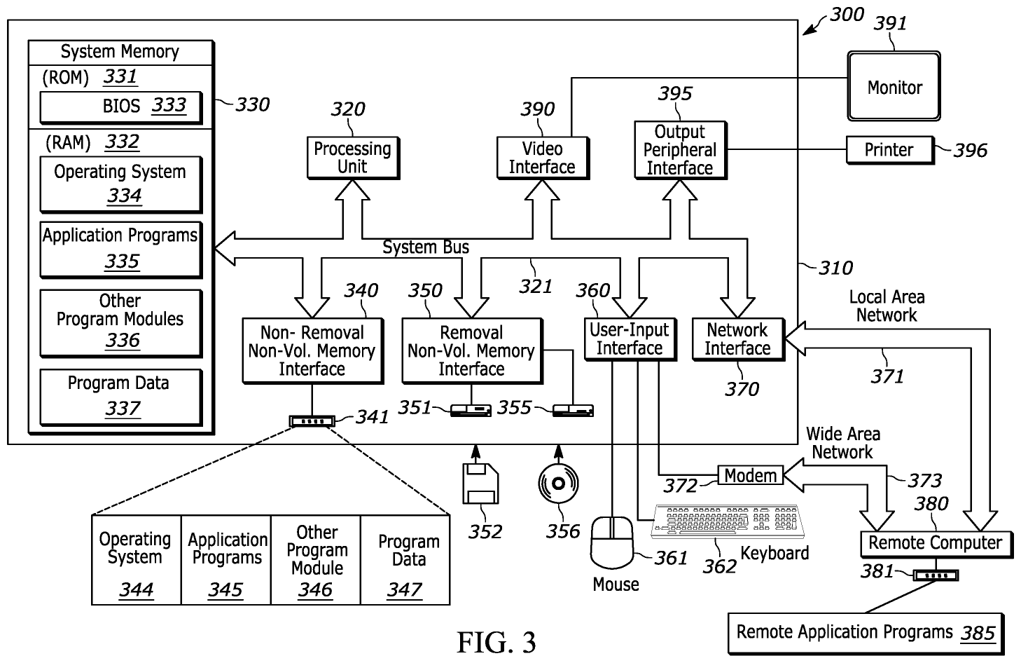

The system 100 may include a mobile computing device 102 (which may include, e.g., a smart phone, a smart watch or fitness tracker device, a tablet, a laptop, a virtual reality headset, smart or augmented reality glasses, wearables, etc.), a computing system 104 (which is described in greater detail below with respect to FIG. 3), and/or one or more home computing device 113 associated with respective homes 114 and home sensors 116. The mobile computing device 102, computing system 104, and/or the home computing devices 113 may be configured to communicate with one another via a wired or wireless computer network 106.

The home computing device 113 may include, or may be configured to communicate with, one or more respective sensors 116 associated with a home environment 114. For instance, the sensors 116 may include interior sensors (e.g., including sensors positioned in various rooms of the home) or exterior sensors (e.g., including sensors positioned inside of the home and/or positioned at an exterior wall of the home and configured to capture data associated with a yard, balcony, deck, or patio of the home, and/or sensors positioned external to the home). The sensors 112 may be configured to capture interior and/or exterior sensor data associated with the home environment 114 and/or appliances or components thereof, including image or video data (e.g., captured by one or more cameras), motion data (e.g., captured by one or more motion detectors), audio data (e.g., captured by one or more microphones), movement data (e.g., captured by one or more accelerometers and/or gyroscopes), temperature data (e.g., captured by one or more temperature sensors), humidity data (e.g., captured by one or more humidity sensors), air flow data (e.g., captured by one or more air flow sensors), water flow or other water data (e.g., captured by one or more water sensors or water flow sensors), lightning or other weather conditions (e.g., captured by a lightning detector), connectivity with the mobile device 102 (e.g., captured by one or more Bluetooth beacons, WiFi gateways), thermal data (e.g., captured by one or more infrared sensors), room occupancy (e.g., captured by one or more room occupancy sensors), etc. In some examples, the sensors may be configured to detect opening or closing of doors and/or windows in the home. Furthermore, the sensors 116 may include sensors integrated within or positioned on various home components, home appliances, plumbing fixtures, etc., including but not limited to freezers, refrigerators, water coolers, ice makers, kitchen stoves, ovens, microwave ovens, washing machines, dryers, dishwashers, air conditioners, heaters, furnaces, water heaters, ventilators, toilets, showers, sinks, sump pumps, pool heating and/or filtration equipment, etc.

Moreover, each of the home computing devices 113 may be configured to collect (or may communicate with other devices configured to collect) home operational data. For instance, the home operational data may include indications of home controls and/or operations performed by a resident of the home, usage data, and/or settings adjusted by a home resident for various home components, home appliances, plumbing fixtures, etc., as well as dates and/or times associated with such controls, operations, usage, and/or settings. For instance, the home operational data may include data associated with electricity operations or electricity usage generally, air conditioning operations or adjustment of settings associated therewith, heating operations or adjustment of settings associated therewith, water heating operations or adjustment of settings associated therewith, cooking operations or adjustment of settings associated therewith, plumbing operations or adjustment of settings associated therewith, dish washing operations or adjustment of settings associated therewith, laundry operations or adjustment of settings associated therewith, pool heating and/or filtration operations or adjustment of settings associated therewith, or any other controls, operations, usage, and/or settings adjustments of any of the home appliances, home components, and/or plumbing fixtures discussed above (or any other home appliances, home components, plumbing fixtures, etc.).

The mobile computing device 102 may include one or more sensors 118, one or more cameras 120, a user interface 122 configured to receive input from users and provide interactive displays to users, and one or more processor(s) 124, as well as one or more computer memories 126. In some examples, the one or more sensors 118 and/or the one or more cameras 120 may include any of the sensors described as home sensors 116. Moreover, in some examples, data captured by the one or more sensors and/or the one or more cameras 120 may be used in addition to or as an alternative to any of data described as being captured by the home sensors 116 above.

Memories 126 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 126 may store an operating system (OS) (e.g., iOS, Microsoft Windows, Linux, UNIX, etc.) capable of facilitating the functionalities, apps, methods, or other software as discussed herein. Memorie(s) 126 may also store a health condition application 128.

Executing the health condition application 128 may include monitoring, (including receiving and/or otherwise obtaining) the sensor data captured by the home sensors 116, mobile device sensors 118, and/or mobile device cameras 120 over various periods of time, and analyzing the sensor data, and/or home operational data over the various periods of time. In particular, executing the health condition application 128 may include analyzing the sensor data associated with the home environment 114 (e.g., including the sensor data from the home sensors 116, mobile device sensors 118, and/or mobile device cameras 120) and/or the operational data associated with the home environment 114 over a first period of time in order to identify a health condition associated with a resident of the home environment.

For example, water or humidity sensors can be used to detect potential mold which may affect health of inhabitants. Additionally, room occupancy sensors, motion sensors, the motion of user mobile devices, smart doorbells and/or front door cameras, etc., may be used to determine whether inhabitants are home, and when inhabitants move throughout the home, in order to determine activity levels and activity times (or lack of activity) that could be indicative of various health conditions. For instance, a lack of motion or leaving the home could be indicative of certain mental or physical health conditions, and motion and/or leaving the home at odd times of night could indicate different health conditions, including mental health issues such as depression or dementia.

Furthermore, data captured by microphones or other sound sensors may determine whether water is running, appliances are being used, people are talking or moving, whether (and in some cases what) people are cooking and/or eating and at what times, any of which may be indicative of various health conditions, especially in combination with other captured data. Moreover, data captured by smart appliances, such as smart refrigerators, or smart ovens or cooktops, may be used to determine cooking/eating habits of individuals, which may be indicative of various health conditions. Additionally, temperature sensors and/or thermal sensors may be used to detect health conditions related to unsafe room temperatures (high or low), or fevers associated with residents of the home environment. Furthermore, electricity sensors may be used to detect power outages, which may result in health conditions based upon the amount of time that the power is out, the ages of the individuals in the home, and the outdoor temperature (e.g., based upon food spoilage, or excessive heat or cold).

As another example, internal cameras may be used as child or infant monitors, and data captured by such internal cameras may be used to identify health conditions associated with children or infants in the home environment. Moreover, in some examples, data from other (e.g., external) sources, such as pollen count, pollution count, UV index, etc., associated with the location of the home environment, may be analyzed in conjunction with the sensor data and/or operational data in order to identify health conditions associated with residents of the home environment 114.

In some examples, identifying the health condition associated with a particular resident of the home environment 114 may be further based upon other data associated with the resident of the home environment 114. For instance, previously-identified health conditions or health data associated with the resident of the home environment (e.g., from an electronic medical record associated with the resident, accessed with permission of the resident) may be analyzed in order to identify health conditions that may be of particular concern to the particular resident. For example, residents with certain pre-existing health conditions, such as residents who are immunocompromised, may be more sensitive to changes in temperature.

In some examples, analyzing the sensor data associated with the home environment 114 (and/or the operational data associated with the home environment 114, and/or the external data) over the first period of time in order to identify the health condition associated with the resident of the home environment 114 may include applying a trained machine learning model to the sensor data associated with the home environment 114 (and/or the operational data associated with the home environment 114) over the period of time in order to identify the health condition associated with the resident of the home environment 114 over the first period of time, e.g., by sending the sensor data (and/or operational data) to the computing system 104, on which a trained machine learning model 138 may be executing (described in greater detail below), and by receiving an identification or prediction of the health condition associated with the resident of the home environment 114 over the first period of time from the computing system 104.

Executing the health condition application 128 may include providing an alert related to the identified health condition to a mobile device 102 associated with the resident of the home environment 114, e.g., audibly or visibly via the user interface 122. Depending on the severity of any identified health conditions associated with the resident of the home environment 114, executing the health condition application 128 may include automatically contacting emergency service providers (e.g., ambulance, fire department, police), and/or automatically contacting medical providers associated with the resident of the home environment 114.

Furthermore, executing the health condition application 128 may include identifying one or more mitigation techniques for any identified health conditions associated with the resident of the home environment 114 over the first period of time. For example, for a health condition associated with mold in the home environment 114, a mitigation technique may include reducing humidity in the home environment 114 or otherwise clearing a water condition in the home environment 114 in order to reduce the amount of mold in the home environment 114. As another example, for a health condition associated with a lack of movement or a lack of leaving the home environment 114, a mitigation technique may include additional daily movement, and possibly a daily movement goal.

As still another example, for a health condition associated with the types of foods consumed by the resident of the home environment 114, the mitigation technique may include switching from packaged or delivered food items to home cooked food items. Furthermore, as another example, for a health condition associated with high or low room temperatures, the mitigation technique may include adjusting the settings of a heating or cooling system in the home environment 114, or repairing a broken heating or cooling system in the home environment 114. Additionally, for a health condition associated with high body temperatures, the mitigation technique may include taking a fever-reducing medication, or scheduling a doctor's appointment for the resident of the home environment 114.

Moreover, executing the health condition application 128 may include providing an indication of any identified mitigation techniques to the residents of the home environment 114 (e.g., audibly or visibly via the user interface 122), or to emergency contacts, family members, or caregivers associated with the residents of the home environment 114, via respective devices associated with those individuals.

Additionally, executing the health condition application 128 may include analyzing the sensor data associated with the home environment 114 (e.g., including the sensor data from the home sensors 116, mobile device sensors 118, and/or mobile device cameras 120) and/or the operational data associated with the home environment 114 over a second period of time, subsequent to providing the indication of the identified mitigation techniques, in order to determine whether any of the identified mitigation techniques have been performed over the second period of time.

In some examples, depending on the severity of any identified health conditions associated with the resident of the home environment 114, executing the health condition application 128 may include automatically contacting emergency service providers (e.g., ambulance, fire department, police), and/or automatically contacting medical providers associated with the resident of the home environment 114 if none of the identified mitigation techniques have been performed over the second period of time.

Furthermore, in some examples, executing the health condition application 128 may include analyzing the sensor data associated with the home environment 114 (e.g., including the sensor data from the home sensors 116, mobile device sensors 118, and/or mobile device cameras 120) and/or the operational data associated with the home environment 114 over a third period of time, subsequent to determining that one or more of the identified mitigation techniques have been performed, in order to determine whether any previously-identified health conditions have changed over the third period of time.

For instance, in some examples, executing the health condition application 128 may include automatically updating a health insurance or life insurance policy associated with the resident of the home environment 114, and/or provide a discount on a health insurance or life insurance policy associated with the resident of the home environment 114, based upon changes in the health condition of the resident of the home environment 114.

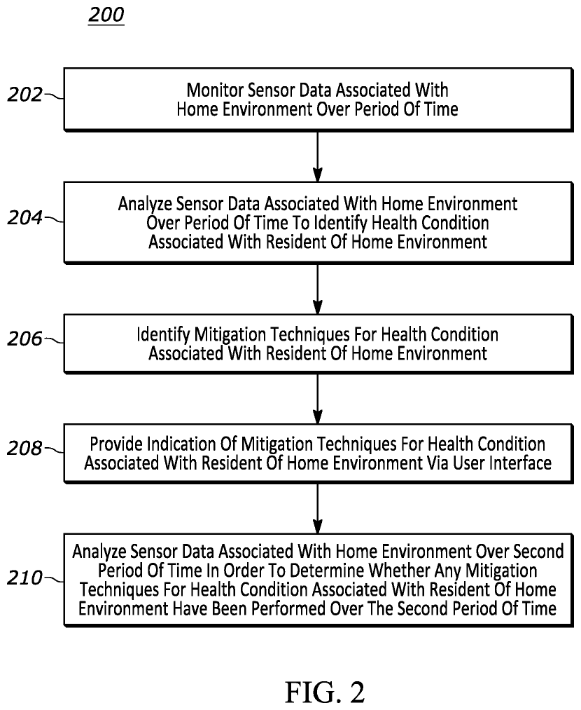

Moreover, in some examples, the computer-readable instructions stored on the memory 126 may include instructions for carrying out any of the steps of the methods 200 via an algorithm executing on the processors 124, which are described in greater detail below with respect to FIG. 2.

In some embodiments the computing system 104 may comprise one or more servers, which may comprise multiple, redundant, or replicated servers as part of a server farm. In still further aspects, such server(s) may be implemented as cloud-based servers, such as a cloud-based computing platform. For example, such server(s) may be any one or more cloud-based platform(s) such as MICROSOFT AZURE, AMAZON AWS, or the like. Such server(s) may include one or more processor(s) 130 (e.g., CPUs) as well as one or more computer memories 132.

Memories 132 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 132 may store an operating system (OS) (e.g., Microsoft Windows, Linux, UNIX, etc.) capable of facilitating the functionalities, apps, methods, or other software as discussed herein. Memorie(s) 132 may also store a health condition diagnostic application 134, a machine learning model training application 136, and/or a health condition diagnostic machine learning model 138.

Additionally, or alternatively, the memorie(s) 132 may store historical health condition diagnostic data. The historical health condition diagnostic data may include historical sensor data or operational data associated with historical home environments over historical periods of time, as well as identified historical health conditions associated with residents of the historical home environments over the historical periods of time. The historical home condition diagnostic data may also be stored in a historical health condition diagnostic database 140, which may be accessible or otherwise communicatively coupled to the computing system 104. In some embodiments, the historical health condition diagnostic data, or other data from various sources may be stored on one or more blockchains or distributed ledgers.

Executing the health condition diagnostic application 134 may include receiving sensor data and/or operational data associated with the home environment over a period of time from the health condition application 128 of the mobile device 102, applying a trained health condition diagnostic machine learning model 138 to the sensor data and/or operational data from the period of time in order to identify health conditions associated with residents of the home environment over the period of time, and/or mitigation techniques associated therewith, and sending indications of the health conditions associated with the residents of the home environment and/or the mitigation techniques associated therewith to the health condition application 128 of the mobile device 102.

In some examples, the trained health condition diagnostic machine learning model 138 may be executed on the computing system 104, while in other examples the health condition diagnostic machine learning model 138 may be executed on another computing system, separate from the computing system 104. For instance, the computing system 104 may send the sensor data and/or operational data associated with the home environment over the period of time from the mobile device 102 to another computing system, where the trained health condition diagnostic machine learning model 138 is applied to the sensor data and/or operational data associated with the home environment over the period of time, and the other computing system may send a prediction or identification of health conditions associated with residents of the home environment, and/or mitigation techniques associated therewith, based upon applying the trained health condition diagnostic machine learning model 138 to the sensor data and/or the operational data associated with the home environment over the period of time, to the computing system 104. Moreover, in some examples, the health condition diagnostic machine learning model 138 may be trained by a machine learning model training application 136 executing on the computing system 104, while in other examples, the health condition diagnostic machine learning model 138 may be trained by a machine learning model training application executing on another computing system, separate from the computing system 104.

Whether the health condition diagnostic machine learning model 138 is trained on the computing system 104 or elsewhere, the health condition diagnostic machine learning model 138 may be trained by the machine learning model training application 136 using training data corresponding to historical sensor data and/or historical operational data associated with home environments over historical periods of time, and historical health conditions associated with the residents of the home environment over the historical periods of time, and/or successful/unsuccessful historical mitigation techniques for the historical health conditions. The trained machine learning model may then be applied to new sensor data and/or new operational data over a new period of time in order to identify or predict, e.g., new health conditions associated with residents of a new home environment over the new period of time, and/or mitigation techniques for the new health conditions.

In various aspects, the health condition machine learning model 138 may comprise a machine learning program or algorithm that may be trained by and/or employ a neural network, which may be a deep learning neural network, or a combined learning module or program that learns in one or more features or feature datasets in particular area(s) of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques.

In some embodiments, the artificial intelligence and/or machine learning based algorithms used to train the health condition machine learning model 138 may comprise a library or package executed on the computing system 104 (or other computing devices not shown in FIG. 1). For example, such libraries may include the TENSORFLOW based library, the PYTORCH library, and/or the SCIKIT-LEARN Python library.

Machine learning may involve identifying and recognizing patterns in existing data (such as training a model based upon historical sensor data and/or operational data associated with a home environment over the period of time and health conditions associated with residents of the home environment and/or mitigation techniques thereof) in order to facilitate making predictions or identification for subsequent data (such as using the machine learning model on new sensor data and/or operational data in order to determine a prediction or identification of new health conditions associated with residents of a home environment and/or mitigation techniques thereof based upon new sensor data and/or operational data associated with the home environment).

Machine learning model(s) may be created and trained based upon example data (e.g., “training data”) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based upon the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. The disclosures herein may use one or both of such supervised or unsupervised machine learning techniques.

In addition, memories 132 may also store additional machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memory 132 may include instructions for carrying out any of the steps of the method 200 via an algorithm executing on the processors 130, which are described in greater detail below with respect to FIG. 2. It should be appreciated that one or more other applications may be envisioned and that are executed by the processor(s) 130. It should be appreciated that given the state of advancements of mobile computing devices, all of the processes functions and steps described herein may be present together on a mobile computing device, such as the mobile computing device 102.

Exemplary Computer-Implemented Method for Detecting Health Conditions Associated with Residents of a Home Environment Based Upon Data Captured by In-Home Sensors

FIG. 2 depicts a flow diagram of an exemplary computer-implemented method 200 for detecting health conditions associated with residents of a home environment based upon data captured by in-home sensors, according to one embodiment. One or more steps of the method 200 may be implemented as a set of instructions stored on a computer-readable memory (e.g., memory 126, memory 132, etc.) and executable on one or more processors (e.g., processor 124, processor 130, etc.).

The method may begin when sensor data associated with a home environment is monitored (block 202). In some examples, operational data associated with the home environment may be monitored as well. In some examples, the sensor data associated with the resident of the home environment may include data captured by sensors of a mobile computing device associated with the resident of the home environment. Furthermore, in some examples, the sensor data associated with the resident of the home environment may include data captured by sensors associated with a vehicle owned or operated by the resident of the home environment. Additionally, in some examples, operational data associated with a vehicle owned or operated by the resident of the home environment may be obtained.

The sensor data (and/or operational data) associated with the home environment over a first period of time may be analyzed (block 204) in order to identify a health condition associated with a resident of the home environment. In some examples, analyzing the sensor data associated with the home environment over the first period of time in order to identify the health condition associated with the resident of the home environment may include applying a trained machine learning model to the sensor data associated with the home environment over the first period of time in order to identify the health condition associated with the resident of the home environment.

For instance, the method 200 may include obtaining historical sensor data associated with historical home environments over historical periods of time, and historical health conditions associated with residents of the historical home environments over the historical periods of time, and the machine learning model may be trained to identify new health conditions associated with residents of new home environments over new periods of time based upon new sensor data associated with the new home environments over the new periods of time, based upon the historical sensor data associated with historical home environments over historical periods of time, and historical health conditions associated with residents of the historical home environments over the historical periods of time, resulting in the trained machine learning model.

One or more mitigation techniques for the health condition associated with the resident of the home environment may be identified (block 206). The mitigation techniques and health condition(s) may include those mentioned elsewhere herein, and/or additional or alternate mitigation techniques and health conditions.

An indication of the one or more mitigation techniques for the health condition associated with the resident of the home environment may be provided (block 208) via a user interface.

The sensor data (and/or operational data) associated with the home environment may be analyzed (block 210) over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time.

In some examples, the method 200 may further include analyzing the sensor data (and/or operational data) associated with the home environment over a third period of time, subsequent to determining that one or more of the mitigation techniques for the health condition associated with the resident of the home environment were performed, in order to identify a change in the health condition associated with the resident of the home environment. In some examples, analyzing the sensor data associated with the home environment over the third period of time in order to identify the change in the health condition associated with the resident of the home environment may include applying a trained machine learning model (e.g., the model discussed with respect to block 204, or a new model) to the sensor data associated with the home environment over the third period of time in order to identify the change in the health condition associated with the resident of the home environment.

Furthermore, in some examples, the method 200 may further include generating an alert related to the health condition associated with the resident of the home environment and sending the alert related to the health condition associated with the resident of the home environment to a medical provider or to a provider of emergency services.

Exemplary Computing System for Detecting Health Conditions Associated with Residents of a Home Environment Based Upon Data Captured by In-Home Sensors

FIG. 3 depicts an exemplary computing system 104 in which the techniques described herein may be implemented, according to one embodiment. The computing system 104 of FIG. 3 may include a computing device in the form of a computer 310. Components of the computer 310 may include, but are not limited to, a processing unit 320 (e.g., corresponding to the processor 120 of FIG. 1), a system memory 330 (e.g., corresponding to the memory 122 of FIG. 1), and a system bus 321 that couples various system components including the system memory 330 to the processing unit 320. The system bus 321 may be any of several types of bus structures including a memory bus or memory controller, a peripheral bus, or a local bus, and may use any suitable bus architecture. By way of example, and not limitation, such architectures include the Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus (also known as Mezzanine bus).

Computer 310 may include a variety of computer-readable media. Computer-readable media may be any available media that can be accessed by computer 310 and may include both volatile and nonvolatile media, and both removable and non-removable media. By way of example, and not limitation, computer-readable media may comprise computer storage media and communication media.

Computer storage media may include volatile and nonvolatile, removable and non-removable media implemented in any method or technology for storage of information such as computer-readable instructions, data structures, program modules or other data. Computer storage media may include, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 310.

Communication media typically embodies computer-readable instructions, data structures, program modules or other data in a modulated data signal such as a carrier wave or other transport mechanism, and may include any information delivery media. The term “modulated data signal” means a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared and other wireless media. Combinations of any of the above are also included within the scope of computer-readable media.

The system memory 330 may include computer storage media in the form of volatile and/or nonvolatile memory such as read only memory (ROM) 331 and random access memory (RAM) 332. A basic input/output system 333 (BIOS), containing the basic routines that help to transfer information between elements within computer 310, such as during start-up, is typically stored in ROM 331. RAM 332 typically contains data and/or program modules that are immediately accessible to, and/or presently being operated on, by processing unit 320. By way of example, and not limitation, FIG. 3 illustrates operating system 334, application programs 335 (e.g., corresponding to the health condition diagnostic application 134, machine learning model training application 136, health condition diagnostic machine learning model 138, etc.), other program modules 336, and program data 337.

The computer 310 may also include other removable/non-removable, volatile/nonvolatile computer storage media. By way of example only, FIG. 3 illustrates a hard disk drive 341 that reads from or writes to non-removable, nonvolatile magnetic media, a magnetic disk drive 351 that reads from or writes to a removable, nonvolatile magnetic disk 352, and an optical disk drive 355 that reads from or writes to a removable, nonvolatile optical disk 356 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 341 may be connected to the system bus 321 through a non-removable memory interface such as interface 340, and magnetic disk drive 351 and optical disk drive 355 may be connected to the system bus 321 by a removable memory interface, such as interface 350.

The drives and their associated computer storage media discussed above and illustrated in FIG. 3 provide storage of computer-readable instructions, data structures, program modules and other data for the computer 310. In FIG. 3, for example, hard disk drive 341 is illustrated as storing operating system 344, application programs 345, other program modules 346, and program data 347. Note that these components may either be the same as or different from operating system 334, application programs 335, other program modules 336, and program data 337. Operating system 344, application programs 345, other program modules 346, and program data 347 are given different numbers here to illustrate that, at a minimum, they are different copies. A user may enter commands and information into the computer 310 through input devices such as cursor control device 361 (e.g., a mouse, trackball, touch pad, etc.) and keyboard 362. A monitor 391 or other type of display device is also connected to the system bus 321 via an interface, such as a video interface 390. In addition to the monitor, computers may also include other peripheral output devices such as printer 396, which may be connected through an output peripheral interface 395.

The computer 310 may operate in a networked environment using logical connections to one or more remote computers, such as a remote computer 380. The remote computer 380 may be a mobile computing device, personal computer, a server, a router, a network PC, a peer device or other common network node, and may include many or all of the elements described above relative to the computer 310, although only a memory storage device 381 has been illustrated in FIG. 3. The logical connections depicted in FIG. 3 include a local area network (LAN) 371 and a wide area network (WAN) 373 (e.g., either or both of which may correspond to the network 108 of FIG. 1), but may also include other networks. Such networking environments are commonplace in hospitals, offices, enterprise-wide computer networks, intranets and the Internet.

When used in a LAN networking environment, the computer 310 is connected to the LAN 371 through a network interface or adapter 370. When used in a WAN networking environment, the computer 310 may include a modem 372 or other means for establishing communications over the WAN 373, such as the Internet. The modem 372, which may be internal or external, may be connected to the system bus 321 via the input interface 360, or other appropriate mechanism. The communications connections 370, 372, which allow the device to communicate with other devices, are an example of communication media, as discussed above. In a networked environment, program modules depicted relative to the computer 310, or portions thereof, may be stored in the remote memory storage device 381. By way of example, and not limitation, FIG. 3 illustrates remote application programs 385 as residing on memory device 381.

The techniques for detecting health conditions associated with residents of a home environment based upon data captured by in-home sensors described above may be implemented in part or in their entirety within a computing system such as the computing system 104 illustrated in FIG. 3. In some such embodiments, the LAN 371 or the WAN 373 may be omitted. Application programs 335 and 345 may include a software application (e.g., a web-browser application) that is included in a user interface, for example.

### Additional Considerations

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” or “some embodiments” means that a particular element, feature, structure, or characteristic described in connection with the embodiment is included in at least one embodiment. The appearances of the phrase “in one embodiment” or “in some embodiments” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for detecting health conditions associated with residents of a home environment based upon data captured by in-home sensors. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A computer-implemented method, comprising:
monitoring, by one or more processors, sensor data associated with a home environment;
analyzing, by one or more processors, the sensor data associated with the home environment over a first period of time in order to identify a health condition associated with a resident of the home environment;
identifying, by the one or more processors, one or more mitigation techniques for the health condition associated with the resident of the home environment;
providing, by the one or more processors, an indication of the one or more mitigation techniques for the health condition associated with the resident of the home environment, via a user interface; and
analyzing, by one or more processors, the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time.

2. The computer-implemented method of claim 1, further comprising:
analyzing, by one or more processors, the sensor data associated with the home environment over a third period of time, subsequent to determining that one or more of the mitigation techniques for the health condition associated with the resident of the home environment were performed, in order to identify a change in the health condition associated with the resident of the home environment.

3. The computer-implemented method of claim 1, wherein monitoring the sensors data associated with the home environment and analyzing the sensor data associated with the home environment includes monitoring operational data associated with the home environment and analyzing the operational data associated with the home environment.

4. The computer-implemented method of claim 1, further comprising:
generating, by the one or more processors, an alert related to the health condition associated with the resident of the home environment; and
sending, by the one or more processors, the alert related to the health condition associated with the resident of the home environment to a medical provider or to a provider of emergency services.

5. The computer-implemented method of claim 1, wherein analyzing the sensor data associated with the home environment over the first period of time in order to identify the health condition associated with the resident of the home environment includes applying a trained machine learning model to the sensor data associated with the home environment over the first period of time in order to identify the health condition associated with the resident of the home environment.

6. The computer-implemented method of claim 5, further comprising:
obtaining, by the one or more processors, historical sensor data associated with historical home environments over historical periods of time, and historical health conditions associated with residents of the historical home environments over the historical periods of time;
and training, by the one or more processors, a machine learning model to identify new health conditions associated with residents of new home environments over new periods of time based upon new sensor data associated with the new home environments over the new periods of time, based upon the historical sensor data associated with historical home environments over historical periods of time, and historical health conditions associated with residents of the historical home environments over the historical periods of time, resulting in the trained machine learning model.

7. A computer system, comprising one or more processors, and a memory storing non-transitory computer-readable instructions that when executed by the one or more processors, cause the one or more processors to:
monitor sensor data associated with a home environment;
analyze the sensor data associated with the home environment over a first period of time in order to identify a health condition associated with a resident of the home environment;
identify one or more mitigation techniques for the health condition associated with the resident of the home environment;
provide an indication of the one or more mitigation techniques for the health condition associated with the resident of the home environment, via a user interface; and
analyze the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time.

8. The computer system of claim 7, wherein the computer-readable instructions, when executed by the one or more processors, further cause the one or more processors to:
analyze the sensor data associated with the home environment over a third period of time, subsequent to determining that one or more of the mitigation techniques for the health condition associated with the resident of the home environment were performed, in order to identify a change in the health condition associated with the resident of the home environment.

9. The computer system of claim 7, wherein monitoring the sensors data associated with the home environment and analyzing the sensor data associated with the home environment includes monitoring operational data associated with the home environment and analyzing the operational data associated with the home environment.

10. The computer system of claim 7, wherein the computer-readable instructions, when executed by the one or more processors, further cause the one or more processors to:
generate an alert related to the health condition associated with the resident of the home environment; and
send the alert related to the health condition associated with the resident of the home environment to a medical provider or to a provider of emergency services.

11. The computer system of claim 7, wherein analyzing the sensor data associated with the home environment over the first period of time in order to identify the health condition associated with the resident of the home environment includes applying a trained machine learning model to the sensor data associated with the home environment over the first period of time in order to identify the health condition associated with the resident of the home environment.

12. The computer system of claim 11, wherein the computer-readable instructions, when executed by the one or more processors, further cause the one or more processors to:
obtain historical sensor data associated with historical home environments over historical periods of time, and historical health conditions associated with residents of the historical home environments over the historical periods of time;
and train a machine learning model to identify new health conditions associated with residents of new home environments over new periods of time based upon new sensor data associated with the new home environments over the new periods of time, based upon the historical sensor data associated with historical home environments over historical periods of time, and historical health conditions associated with residents of the historical home environments over the historical periods of time, resulting in the trained machine learning model.

13. A computer-readable medium storing non-transitory computer-readable instructions that, when executed by one or more processors, cause the one or more processors to:
monitoring, by one or more processors, sensor data associated with a home environment;
analyzing, by one or more processors, the sensor data associated with the home environment over a first period of time in order to identify a health condition associated with a resident of the home environment;
identifying, by the one or more processors, one or more mitigation techniques for the health condition associated with the resident of the home environment;
providing, by the one or more processors, an indication of the one or more mitigation techniques for the health condition associated with the resident of the home environment, via a user interface;
analyzing, by one or more processors, the sensor data associated with the home environment over a second period of time, subsequent to providing the indication of the one or more mitigation techniques for the health condition associated with the resident of the home environment via the user interface, in order to determine whether any of the one or more mitigation techniques for the health condition associated with the resident of the home environment were performed over the second period of time.

14. The computer-readable medium of claim 13, wherein the computer-readable instructions, when executed by the one or more processors, further cause the one or more processors to:
analyze the sensor data associated with the home environment over a third period of time, subsequent to determining that one or more of the mitigation techniques for the health condition associated with the resident of the home environment were performed, in order to identify a change in the health condition associated with the resident of the home environment.

15. The computer-readable medium of claim 13, wherein monitoring the sensors data associated with the home environment and analyzing the sensor data associated with the home environment includes monitoring operational data associated with the home environment and analyzing the operational data associated with the home environment.

16. The computer-readable medium of claim 13, wherein the computer-readable instructions, when executed by the one or more processors, further cause the one or more processors to:
generate an alert related to the health condition associated with the resident of the home environment; and
send the alert related to the health condition associated with the resident of the home environment to a medical provider or to a provider of emergency services.

17. The computer-readable medium of claim 13, wherein analyzing the sensor data associated with the home environment over the first period of time in order to identify the health condition associated with the resident of the home environment includes applying a trained machine learning model to the sensor data associated with the home environment over the first period of time in order to identify the health condition associated with the resident of the home environment.

18. The computer-readable medium of claim 17, wherein the computer-readable instructions, when executed by the one or more processors, further cause the one or more processors to:
obtain historical sensor data associated with historical home environments over historical periods of time, and historical health conditions associated with residents of the historical home environments over the historical periods of time;
and train a machine learning model to identify new health conditions associated with residents of new home environments over new periods of time based upon new sensor data associated with the new home environments over the new periods of time, based upon the historical sensor data associated with historical home environments over historical periods of time, and historical health conditions associated with residents of the historical home environments over the historical periods of time, resulting in the trained machine learning model.
